(19) United States
(12) Patent Application Publication
(10) Pub. No.: US 2026/0158294 A1
(43) Pub. Date: Jun. 11, 2026

(54) Imaging Method, Imaging System, Electronic Device and Storage Medium

(71) Applicant: OUR UNITED CORPORATION, Shaanxi (CN)

(72) Inventors: Hao Yan, Shaanxi (CN); Dalin Liu, Shaanxi (CN)

(21) Appl. No.: 19/180,338

(22) Filed: Apr. 16, 2025

(30) Foreign Application Priority Data
Apr. 17, 2024 (CN) 202410465016.3

(51) Int. Cl. A61N 5/10

(52) U.S. Cl. CPC A61N 5/1037; A61N 5/1039

(57) ABSTRACT

The present disclosure provides an imaging method and an imaging system, including a multi-focal tube and a detector arranged opposite to the multi-focal tube. The method includes grouping focal light sources in the multi-focal tube to obtain a plurality of focal light source groups and controlling the multi-focal tube to sequentially expose the plurality of focal light source groups, each of which includes at least one focal light source; acquiring sequentially, from the detector, a projection image of a target soft tissue exposed by each focal light source group, where the projection image includes a sub-projection image of the target soft tissue irradiated by each focal light source in the focal light source group corresponding to the projection image; and reconstructing a plurality of sub-projection images in the projection image corresponding to each focal light source group to obtain a three-dimensional image of the target soft tissue.

### CROSS REFERENCE TO RELATED APPLICATION

This application claims priority to Chinese Patent Application No. 202410465016.3, filed April 17, 2024, the disclosure of which is hereby incorporated by reference in its entirety.

### BACKGROUND OF THE INVENTION

### FIELD OF THE INVENTION

The present disclosure relates to the field of medical technologies, in particular to the field of soft tissue imaging technologies, and specifically to an imaging method, an imaging system, an electronic device and a storage medium.

### DESCRIPTION OF RELATED ART

In the field of medical technologies, radiation therapy (abbreviated as radiotherapy) is one of the important means of treating tumors. Whether during the image-guided setting up phase for aligning a target volume (soft tissue) or during the image-guided radiation therapy monitoring phase for imaging and tracking the target volume, the imaging is expected to satisfy both “real-time” and “three-dimensional” requirements.

Currently, existing image generating methods are usually based on the cone beam computed tomography (CBCT) technology or the kilovolt (KV) beam technology for imaging. However, the CBCT technology requires rotational imaging during the three-dimensional imaging process, which results in relatively slow imaging speeds, and fails to meet the “real-time” requirement. Meanwhile, a projection image generated by the KV beam technology during the projection imaging process is a two-dimensional image, in which only the bone skeleton is relatively clear, but a three-dimensional structure of the soft tissue cannot be displayed clearly, which fails to meet the “three-dimensional” requirement.

### SUMMARY OF THE INVENTION

The present disclosure provides an imaging method, an imaging system, an electronic device and a storage medium, which may quickly and accurately obtain a three-dimensional image of a target soft tissue.

In a first aspect, the present disclosure provides an imaging method for controlling an imaging system to perform imaging. The imaging system includes a multi-focal tube and a detector arranged opposite to the multi-focal tube; the multi-focal tube includes a plurality of focal light sources; the imaging method includes: grouping the focal light sources in the multi-focal tube to obtain a plurality of focal light source groups and controlling the multi-focal tube to sequentially expose the plurality of focal light source groups, where each focal light source group of the plurality of focal light source groups includes at least one focal light source; acquiring sequentially, from the detector, a projection image of a target soft tissue exposed by each focal light source group, where the projection image includes a sub-projection image of the target soft tissue irradiated by each focal light source in the focal light source group corresponding to the projection image, and image contents of sub-projection images in the projection image do not overlap; and reconstructing a plurality of sub-projection images in the projection image corresponding to each focal light source group to obtain a three-dimensional image of the target soft tissue.

In a second aspect, the present disclosure provides an imaging system, including: a gantry; a multi-focal tube, the multi-focal tube including a plurality of focal light sources; a detector arranged opposite to the multi-focal tube; and an imaging control processing device, configured to perform any imaging method in the above first aspect.

In a third aspect, the present disclosure further provides an electronic device. The electronic device includes: a processor and a memory configured to store instructions executable by the processor; where the processor is configured to execute the instructions to implement any imaging method in the above first aspect.

In a fourth aspect, the present disclosure further provides a non-volatile storage medium having a computer program stored on the storage medium, and when the computer program is read and executed, any imaging method in the above first aspect is implemented.

In the imaging method provided by the present disclosure, the imaging control processing device may group the focal light sources in the multi-focal tube, and control the multi-focal tube to sequentially expose respective focal light source groups (including at least one focal light source). Since the imaging control processing device sequentially exposes the respective focal light source groups in the form of groups, the imaging control processing device may acquire projection sub-images of a plurality of focal light sources in the respective focal light source groups at one time (i.e., at the same time), thereby improving the efficiency of the imaging.

Secondly, since the respective focal light sources irradiate the target soft tissue from different directions, the imaging control processing device acquires sequentially, from the detector, the projection image of the target soft tissue exposed by each focal light source group, which also includes sub-projection images acquired from the different directions (i.e., a plurality of different angles). By reconstructing a plurality of sub-projection images in the projection image corresponding to each focal light source group, a three-dimensional image of the target soft tissue may be obtained quickly and accurately, which improves the efficiency of the imaging and then may quickly and accurately locate a position of the target soft tissue in three-dimensional space.

### BRIEF DESCRIPTION OF THE DRAWINGS

The drawings are used to better understand the present solutions and do not constitute a limitation on the present disclosure. Herein:

FIG. 1 is a scene schematic diagram of an imaging system, provided by the embodiments of the present disclosure.

FIG. 2 is a flow schematic diagram of an imaging method, provided by the embodiments of the present disclosure.

FIG. 3 is a structural schematic diagram of imaging based on a multi-focal light source tube, provided by the embodiments of the present disclosure.

FIG. 4 is a flow schematic diagram of another imaging method, provided by the embodiments of the present disclosure.

FIG. 5 is a flow schematic diagram of yet another imaging method, provided by the embodiments of the present disclosure.

FIG. 6 is a flow schematic diagram of yet another imaging method, provided by the embodiments of the present disclosure.

FIG. 7 is a flow schematic diagram of yet another imaging method, provided by the embodiments of the present disclosure.

FIG. 8 is a flow schematic diagram of yet another imaging method, provided by the embodiments of the present disclosure.

FIG. 9 is a flow schematic diagram of an imaging control processing apparatus, provided by the embodiments of the present disclosure.

FIG. 10 is a schematic block diagram of an electronic device, provided by the embodiments of the present disclosure.

### DESCRIPTION OF THE INVENTION

The technical solutions in the embodiments of the present disclosure will be described clearly and completely below with reference to the drawings in the embodiments of the present disclosure. Obviously, the described embodiments are merely some but not all of the embodiments of the present disclosure. All other embodiments obtained by those skilled in the art based on the embodiments of the present disclosure without paying any creative effort shall fall within the protection scope of the present disclosure.

In the description of the present disclosure, it should be understood that, orientation or positional relationships indicated by the terms such as "center/central", "length", "width", “upper”, “lower”, etc., are orientation or positional relationships shown based on the drawings, which are merely for convenience in the description of the present disclosure and simplifying the description, but do not indicate or imply that the indicated apparatus or element must have a specific orientation, or must be constructed and operated in a specific orientation, and thus cannot be interpreted as limitations on the present disclosure. In addition, the terms "first", "second" and "third", etc., are merely for the purpose of description and cannot be interpreted as indicating or implying the relative importance or implicitly indicating the number of the indicated technical features. Thus, features defined with "first", "second" and "third", etc., may explicitly or implicitly include one or more of the features. In the description of the present disclosure, the term "a plurality of/multiple" means two or more unless otherwise defined specifically.

In the description of the present disclosure, the word "exemplary/exemplarily" is used to represent "using as an example, instance, or illustration". Any embodiment described as "exemplary/exemplarily" in the present disclosure is not necessarily to be construed as preferred or advantageous over other embodiments. The following description is presented to enable any person skilled in the art to implement and use the present disclosure. In the following description, details are set forth for purposes of explanation. It should be appreciated that those ordinary skilled in the art may recognize that the present disclosure may be implemented without using these specific details. In other instances, well-known structures and processes will not be described in detail, to prevent unnecessary details from obscuring the description of the present disclosure. Thus, the present disclosure is not intended to be limited to the embodiments as shown, but is to be accorded the widest scope consistent with the principles and features disclosed in the present disclosure.

It should be noted that, since the method of the embodiments of the present disclosure is executed in an imaging control processing device, the respective processed objects of the imaging control processing device exist in the form of data or information. For example, time is time information actually. It can be understood that if size, quantity/number, position/location, etc., are mentioned in the following embodiments, it means that data corresponding to them exist, so that the imaging control processing device may process them. Specific details will not be repeated herein.

In the field of medical technologies, radiation therapy (abbreviated as radiotherapy) is one of the important means of treating tumors. Whether during the image-guided setting up phase for setting up a patient to align a target volume (soft tissue) to a reference position, or during graphic-guided radiation therapy process for imaging and tracking the target volume to monitor whether the target volume deviates from the reference position, the imaging is expected to satisfy both “real-time” and “three-dimensional” requirements.

Currently, existing image generating methods are usually based on the CBCT technology or KV beam technology for imaging. However, the CBCT technology requires rotational imaging during the three-dimensional imaging process, which results in relatively slow imaging speeds, and fails to meet the “real-time” requirement. Meanwhile, a projection image generated by the KV beam technology during the projection imaging process is a two-dimensional image, in which only the bone skeleton is relatively clear, but a three-dimensional structure of the soft tissue cannot be displayed clearly, which fails to meet the “three-dimensional” requirement.

Based on the above technical problems, the embodiments of the present disclosure provide an imaging method for controlling an imaging system to perform imaging. FIG. 1 is a scene schematic diagram of an imaging system, provided by the embodiments of the present disclosure, and the imaging system may include: a gantry101, a multi-focal tube 102 arranged on the gantry 101, a detector 103 arranged opposite to the multi-focal tube 102, and an imaging control processing device 104.

Herein, the gantry 101 may be an annular gantry, a C-arm, a drum gantry, a multi-layer bowl-shaped/cylindrical structure gantry, etc., that may support the multi-focal tube 102 and the detector 103. The gantry 101 may be a rotating gantry that may move around a rotation axis or a non-movable fixed gantry.

The multi-focal tube 102 includes a plurality of focal light sources 105 (seven focal light sources are taken as an example for explanation in FIG. 1). The plurality of focal light sources may be arranged linearly or arranged in an arc shape. In a case where the plurality of focal light sources are arranged in an arc shape, a distance between each focal light source and an imaging center of the imaging system is equal or approximately equal.

The rays emitted by the respective focal light sources 105 may include imaging beams (e.g., X-rays), etc. The imaging beam passes through the target volume of the patient and is received by the detector.

In an implementation, the imaging system may further include a plurality of beam limiters 106 corresponding one-to-one to the plurality of focal light sources 105.

The beam limiter 106 is used to beam-limit the rays emitted by the focal light source 105, and an aperture direction and an aperture size of the beam limiter are adjustable.

Exemplarily, when the aperture direction of the beam limiter 106 is a preset direction, the direction of the central ray of the focal light source 105 corresponding to the beam limiter 106 may be made to be the direction of the target soft tissue. When the aperture size of the beam limiter 106 is a preset size, a range error between a soft tissue irradiation range of the focal light source 105 corresponding to the beam limiter 106 and a target volume range of the target soft tissue may be made smaller than the preset range.

In another implementation, the above-mentioned plurality of beam limiters 106 may be replaced with a whole beam limiter. The whole beam limiter may include a plurality of apertures corresponding one-to-one to the plurality of focal light sources 105. In this way, rays emitted by the respective focal light sources 105 may also be beam-limited by the respective apertures.

The detector 103 is used to receive the projection image of the target soft tissue exposed by the respective focal light sources 105.

In the embodiments of the present disclosure, the detector 103 may be a flat-panel detector or a curved-surface detector, and the embodiments of the present disclosure do not limit the detector 103 specifically.

The imaging control processing device 104 is communicatively connected to the multi-focal tube 102 and the detector 103 respectively, and the imaging control processing device is used to control the multi-focal tube 102 to sequentially expose the respective focal light source groups, and acquire sequentially from the detector 103, the projection image of the target soft tissue exposed by each focal light source group, and reconstruct a plurality of sub-projection images in the projection image corresponding to each focal light source group, to obtain a three-dimensional image of the target soft tissue. In some embodiments, the imaging control processing device 104 is a computer device with a graphical user interface (Graphical User Interface, GUI), and the computer device includes: one or more processors, a memory, and one or more application programs. Exemplarily, the imaging control processing device 104 may include an imaging system application program, and a processor of the imaging control processing device executes the imaging system application program to implement: reconstructing a plurality of sub-projection images in the projection image corresponding to each focal light source group, to obtain a three-dimensional image of the target soft tissue.

In some embodiments, the imaging control processing device 104 may also be used to execute one or more of the following operations: controlling the gantry 101 to rotate around a preset axis of the gantry 101, thereby driving the multi-focal tube 102 and the detector 103 to move around the preset axis of the gantry 101; and controlling the multi-focal tube 102 to move closer to or further away from the detector 103.

Herein, the preset axis of the gantry may be a rotation axis of the gantry, and the multi-focal tube 102 and/or the detector 103 may move around the preset axis of the gantry along a track arranged on the gantry 101.

It should be noted that, in a case where the multi-focal tube 102 and the detector 103 are both arranged fixedly on the gantry, the multi-focal tube 102 and the detector 103 move together around the preset axis of the gantry along the track on the gantry 101.

In the embodiments of the present disclosure, an entity of the imaging control processing device 104 may be a terminal or a server, which is not limited to the embodiments of the present disclosure.

In some embodiments, the above-mentioned terminal may be at least one of a smart phone, a smart watch, a desktop computer, a handheld computer, a virtual reality terminal, an augmented reality terminal, a wireless terminal, a laptop portable computer and other devices.

In some embodiments, the above-mentioned server may be an independent physical server, or a server cluster composed of a plurality of physical servers or a distributed file system, or a cloud server that provides at least one of basic cloud computing services such as cloud services, cloud databases, cloud computing, cloud functions, cloud storage, network services, cloud communications, middleware services, domain name services, security services, content distribution networks, and big data or artificial intelligence platforms, which are not limited in the embodiments of the present disclosure. In some embodiments, a number of the above-mentioned servers can be more or less, which is not limited in the embodiments of the present disclosure. Of course, the server can also include other functions to provide more comprehensive and diverse services.

Furthermore, in some embodiments, the imaging control processing device 104 may run a computer system, the computer system includes a processor, and the processor is configured to implement the imaging method provided by the embodiments of the present disclosure.

The imaging method provided by the embodiments of the present disclosure will be introduced below based on the imaging system shown in FIG. 1.

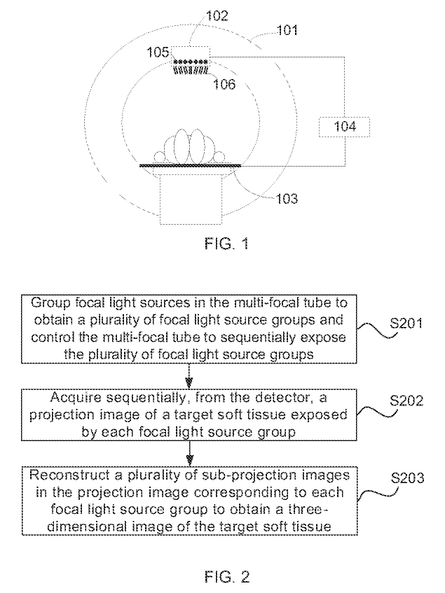

The imaging method provided by the embodiments of the present disclosure is applied to the imaging control processing device 104 in FIG. 1. FIG. 2 shows a flowchart schematic diagram of an imaging method, provided by the embodiments of the present disclosure. As shown in FIG. 2, the imaging method includes: S201 to S203.

S201, the imaging control processing device groups focal light sources in the multi-focal tube to obtain a plurality of focal light source groups and controls the multi-focal tube to sequentially expose the plurality of focal light source groups.

Herein, each focal light source group of the plurality of focal light source groups includes at least one focal light source.

Specifically, in the multi-focal tube, power of the respective focal light sources is usually low, and therefore, exposure time of the respective focal light sources irradiating the soft tissue is usually long. Also, the multi-focal tube usually includes dozens of focal light sources.

Exemplarily, a maximum operating voltage of the above-mentioned respective focal light sources is usually 120 kV, a maximum current thereof is usually 15 mA, and the exposure time for irradiating the soft tissue is usually tens of ms. Also, the multi-focal tube may usually integrate about 40 focal light sources within a length range of 60 cm.

If the imaging control processing device controls each focal light source in the multi-focal tube to be exposed sequentially, the exposure time is long, and correspondingly, the imaging speed is slow. In this case, the imaging control processing device may group the focal light sources in the multi-focal tube and control the multi-focal tube to expose the respective focal light source groups sequentially. In this way, by exposing the respective focal light source groups in the form of groups, the projection sub-images of a plurality of focal light sources in the respective focal light source groups may be acquired at one time, thereby improving the efficiency of the imaging.

In an implementation, in order to ensure the integrity of the projection image of the target soft tissue exposed by each focal light source group and the efficiency of the subsequent three-dimensional imaging, each focal light source group satisfies conditions that: the image contents of the sub-projection images of the target soft tissue irradiated by each focal light source in the focal light source group do not overlap, and a number of focal light sources in the focal light source group is greater than a preset number, and a soft tissue irradiation range of each focal light source in the focal light source group covers the target soft tissue, and a range error between the soft tissue irradiation range of each focal light source in the focal light source group and a soft tissue range of the target soft tissue is less than a preset range.

Herein, a condition that the image contents of the sub-projection images of the target soft tissue irradiated by each focal light source in the focal light source group do not overlap, means that: the image contents of the sub-projection images of the target soft tissue irradiated by each focal light source in the focal light source group on are image contents of different volumes of the target soft tissue. In this way, when the imaging control processing device performs three-dimensional imaging subsequently, the imaging control processing device may perform rapid imaging according to the image contents of different volumes of the target soft tissue.

A condition that the number of focal light sources in the focal light source group is greater than the preset number means that: the number of focal light sources in the focal light source group is as large as possible, on the basis that the image contents of the sub-projection images of the target soft tissue irradiated by each focal light source in the focal light source group do not overlap. In this way, by exposing a plurality of focal light sources simultaneously, the image control processing device may acquire simultaneously the projection sub-images of the plurality of focal light sources in each focal light source group, thereby improving the efficiency of the imaging.

A condition that the soft tissue irradiation range of each focal light source in the focal light source group covers the target soft tissue means that: the soft tissue irradiation range of each focal light source needs to cover the target soft tissue, so that a complete projection image of the target soft tissue can be acquired, thereby improving the accuracy of three-dimensional imaging.

A condition that the range error between the soft tissue irradiation range of each focal light source in the focal light source group and the soft tissue range of the target soft tissue is less than the preset range means that: on the basis that the soft tissue irradiation range of each focal light source in the focal light source group covers the target soft tissue, the soft tissue irradiation range of each focal light source cannot exceed the soft tissue range of the target soft tissue by too much, thereby avoiding imaging errors caused by the soft tissue irradiation range of each focal light source irradiating volumes outside the soft tissue range of the target soft tissue.

In an implementation, the imaging control processing device may group the focal light sources in the multi-focal tube according to geometric data in the imaging system, or may group the focal light sources in the multi-focal tube according to artificial experiences, or may group the focal light sources in the multi-focal tube in other manners, which are not limited in the embodiments of the present disclosure.

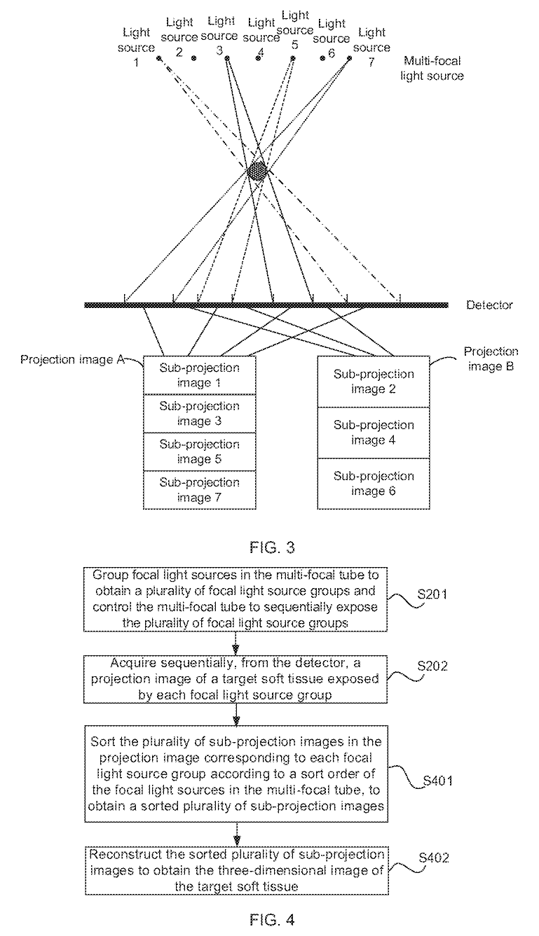

Exemplarily, as shown in FIG. 3, in a case where the focal light sources in the multi-focal tube include light source 1, light source 2, light source 3, light source 4, light source 5, light source 6, and light source 7, the imaging control processing device may group the above 7 focal light sources into 2 groups, which respectively are: a first focal light source group consisting of light source 1, light source 3, light source 5, and light source 7, and a second focal light source group consisting of light source 2, light source 4, and light source 6.

It should be noted that, in order to clearly depict rays of the light sources, FIG. 3 only shows light source rays of the first focal light source group. Light source rays of the second focal light source group are not shown. It can be understood that, the light source rays of the second focal light source group may be inferred by the light source rays of the first focal light source group.

S202, the imaging control processing device acquires sequentially, from the detector, a projection image of a target soft tissue exposed by each focal light source group.

Specifically, after the multi-focal tube is controlled to expose sequentially the respective focal light source groups, the detector may acquire the projection image of the target soft tissue exposed by each focal light source group. The imaging control processing device may acquire sequentially from the detector, the projection image of the target soft tissue exposed by each focal light source group.

Herein, the projection image includes a sub-projection image of the target soft tissue irradiated by each focal light source in the focal light source group corresponding to the projection image. Image contents of sub-projection images in the projection image do not overlap.

Exemplarily, as shown in FIG. 3, after the multi-focal tube is controlled to sequentially expose the first focal light source group and the second focal light source group, the detector may sequentially acquire projection image A of the target soft tissue exposed by the first focal light source group and projection image B of the target soft tissue exposed by the second focal light source group. Correspondingly, the imaging control processing device may acquire sequentially, from the detector, projection image A and projection image B.

Herein, projection image A includes sub-projection image 1 of the target soft tissue irradiated by light source 1, sub-projection image 3 of the target soft tissue irradiated by light source 3, sub-projection image 5 of the target soft tissue irradiated by light source 5, and sub-projection image 7 of the target soft tissue irradiated by light source 7. Projection image B includes sub-projection image 2 of the target soft tissue irradiated by light source 2, sub-projection image 4 of the target soft tissue irradiated by light source 4, and sub-projection image 6 of the target soft tissue irradiated by light source 6.

In an implementation, since the projection image acquired by the imaging control processing device from the detector, may contain noise data, the imaging control processing device, after acquiring the projection image from the detector, may perform pre-processing (e.g., image correction, etc.) on the projection image, to obtain a noise-reduced projection image.

S203, the imaging control processing device reconstructs a plurality of sub-projection images in the projection image corresponding to each focal light source group to obtain a three-dimensional image of the target soft tissue.

Specifically, since the plurality of focal light sources in the multi-focal tube are distributed at different positions, different focal light sources may irradiate the target soft tissue from different directions (angles). Correspondingly, the plurality of sub-projection images in the projection image corresponding to each focal light source group are sub-projection images acquired from a plurality of different directions (angles). In this case, the imaging control processing device reconstructs the plurality of sub-projection images in the projection image corresponding to each focal light source group, so as to obtain a three-dimensional image of the target soft tissue (i.e., three-dimensional imaging is performed by the appropriate geometric design).

It can be understood that, the above-mentioned three-dimensional imaging may also be referred to as tomography.

In some embodiments, the imaging control processing device may reconstruct the plurality of sub-projection images by a preset reconstruction algorithm (e.g., an optical path modeling algorithm), to obtain the three-dimensional image of the target soft tissue.

In an implementation, if the multi-focal tube is fixed in a certain direction (angle) of the target soft tissue, the imaging control processing device may not acquire the projection images of the target soft tissue in various directions (angles), and thus cannot generate a complete three-dimensional image of the target soft tissue. In this case, the imaging control processing device may control the frame on which the multi-focal tube is installed, and/or control the multi-focal tube to move (including at least one of a rotational motion, a translational motion, or an up-down motion), so that the plurality of focal light sources in the multi-focal tube repeatedly irradiate the target soft tissue from different directions (angles), and then projection images of the target soft tissue in various directions (angles) are acquired, so as to generate a complete three-dimensional image of the target soft tissue or a three-dimensional image in a preset direction (angle).

The scheme in which the imaging control processing device generates the three-dimensional image of the target soft tissue in various directions (angles) may refer to the specific description of S201 to S203, which will not be repeated herein.

In an implementation, since it takes a certain amount of time to generate a three-dimensional image of the target soft tissue, therefore, in a scenario where the imaging control processing device generates a three-dimensional image of the target soft tissue in various directions (angles), the imaging control processing device may decouple the process of generating a three-dimensional image of the target soft tissue in a certain direction (i.e., the reconstruction process of three-dimensional imaging) from the process of the imaging control processing device acquiring a projection image of the target soft tissue in another direction (i.e., the image collecting process), thereby performing the above-mentioned reconstruction process of three-dimensional imaging and the image collecting process in parallel, which improves the efficiency of the imaging.

In an implementation, in combination with FIG. 2, as shown in FIG. 4, in the above-mentioned S203, the method for the imaging control processing device to reconstruct the plurality of sub-projection images in the projection image corresponding to each focal light source group to obtain the three-dimensional image of the target soft tissue, specifically includes:

S401, the imaging control processing device sorts the plurality of sub-projection images in the projection image corresponding to each focal light source group according to a sort order of the focal light sources in the multi-focal tube, to obtain a sorted plurality of sub-projection images.

Specifically, since the focal light sources in the multi-focal tube are sorted in order, the plurality of sub-projection images should also be sorted in order. To quickly and accurately generate the three-dimensional image of the target soft tissue by the plurality of sub-projection images sorted in order, the imaging control processing device may acquire the sort order of the focal light sources in the multi-focal tube in advance.

In some embodiments, the sort order may be configured by the user in real time, or may be pre-configured at the factory, which is not limited in the embodiments of the present disclosure.

In an implementation, since the imaging control processing device may generate noise data in the sorted plurality of sub-projection images when sorting the plurality of sub-projection images, the imaging control processing device, after obtaining the sorted plurality of sub-projection images, may perform pre-processing (such as image correction, etc.) on the sorted plurality of sub-projection images, to obtain the noise-reduced and sorted plurality of sub-projection images.

S402, the imaging control processing device reconstructs the sorted plurality of sub-projection images to obtain the three-dimensional image of the target softtissue.

Continuing with the example provided in FIG. 3, it is assumed that the sort order of the seven focal light sources is light source 1, light source 2, light source 3, light source 4, light source 5, light source 6, and light source 7, in sequence. After acquiring projection image A and projection image B, the imaging control processing device may sort the 7 sub-projection images in projection image A and projection image B according to the sort order of the above-mentioned 7 focal light sources, to obtain the sorted plurality of sub-projection images, which sequentially are: sub-projection image 1, sub-projection image 2, sub-projection image 3, sub-projection image 4, sub-projection image 5, sub-projection image 6, and sub-projection image 7.

Next, the imaging control processing device may reconstruct sub-projection image 1, sub-projection image 2, sub-projection image 3, sub-projection image 4, sub-projection image 5, sub-projection image 6, and sub-projection image 7, to obtain the three-dimensional image of the target soft tissue.

In this way, by controlling the exposure order of the plurality of focal light source groups, and sorting and reconstructing the plurality of sub-projection images, the imaging control processing device may quickly and accurately obtain the three-dimensional image of the target soft tissue.

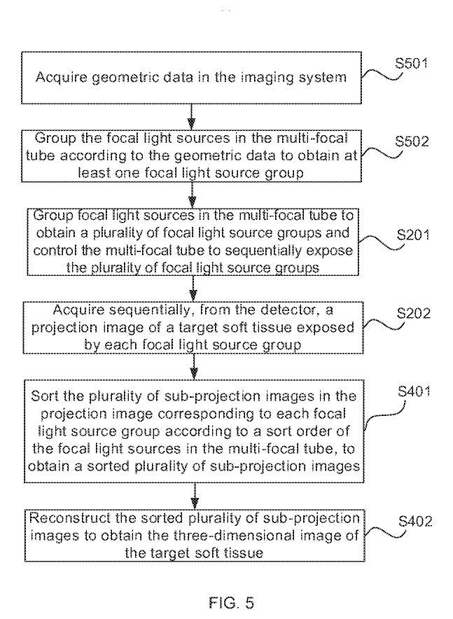

In an implementation, in combination with FIG. 4 and as shown in FIG. 5, in the above-mentioned S201, the method for the imaging control processing device to group the focal light sources in the multi-focal tube, specifically includes:

S501, the imaging control processing device acquires geometric data in the imaging system.

Herein, the geometric data includes: a linear distance between each focal light source and a center point of the target soft tissue, a vertical distance between the target soft tissue and the detector, a shape and a size of the target soft tissue, and a distance between two focal light sources of the plurality of focal light sources (also referred as a distributed width of the focal light sources in the multi-focal tube, such as 60 cm).

S502, the imaging control processing device groups the focal light sources in the multi-focal tube according to the geometric data to obtain at least one focal light source group.

Specifically, it can be seen from the above-mentioned S201 that each focal light source group needs to satisfy certain conditions. Therefore, the imaging control processing device may group the focal light sources in the multi-focal tube according to the geometric data in the imaging system, so that each focal light source group satisfies the respective conditions in the above-mentioned S201.

Exemplarily, since the soft tissue irradiation range of each focal light source is fixed, if the distance between the plurality of focal light sources is short, the soft tissue irradiation ranges of the plurality of focal light sources may overlap, and if the distance between the plurality of focal light sources is far, the soft tissue irradiation ranges of the plurality of focal light sources may not fully cover the target soft tissue. Therefore, by acquiring the distance between two focal light sources of the plurality of focal light sources, the focal light sources in the multi-focal tube may be reasonably grouped, so that the soft tissue irradiation range of each focal light source in the focal light source group covers the target soft tissue, and the range error between the soft tissue irradiation range of each focal light source in the focal light source group and the soft tissue range of the target soft tissue is less than the preset range.

Similarly, the linear distance between each focal light source and the center point of the target soft tissue, the vertical distance between the target soft tissue and the detector, and the shape and the size of the target soft tissue are all related to conditions such as the soft tissue irradiation range of the focal light source and the image content of the sub-projection image of the target soft tissue irradiated by the focal light source. Therefore, the imaging control processing device acquires the geometric data in the imaging system, and may group the focal light sources in the multi-focal tube according to the geometric data reasonably, to obtain at least one focal light source group that satisfies various conditions in the above-mentioned S201.

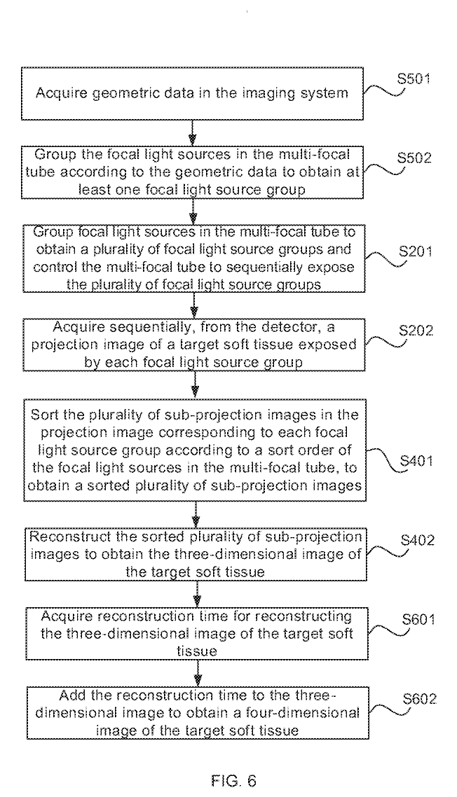

In an implementation, in combination with FIG. 5 and as shown in FIG. 6, the imaging method provided by the embodiments of the present disclosure further includes: S601 to S602.

S601, the imaging control processing device acquires reconstruction time for reconstructing the three-dimensional image of the target soft tissue.

Specifically, when monitoring and/or image-guiding the target soft tissue, the imaging control processing device may reconstruct a three-dimensional image of the target soft tissue at a plurality of time points. Therefore, the imaging control processing device may acquire the reconstruction time for reconstructing the three-dimensional image of the target soft tissue.

In some embodiments, since the time for reconstructing the three-dimensional image of the target soft tissue, acquired by the imaging control processing device, is short, the above-mentioned reconstruction time may be the moment when the reconstructing of the three-dimensional image of the target soft tissue is completed, or the moment when the projection image for the target soft tissue is acquired, or other moments in the reconstruction procedure, which is not limited in the embodiments of the present disclosure.

S602, the imaging control processing device adds the reconstruction time to the three-dimensional image to obtain a four-dimensional image of the target soft tissue.

The above four-dimensional image includes the reconstruction time of the target soft tissue in a time dimension, in addition to the three-dimensional image of the target soft tissue in the physical space. In this way, a physicist may acquire the three-dimensional images of the target soft tissue at different time points by the reconstruction time of the target soft tissue in the time dimension, thereby helping the physicist to monitor the target soft tissue and/or contributing to the accuracy of the image-guiding of the target soft tissue.

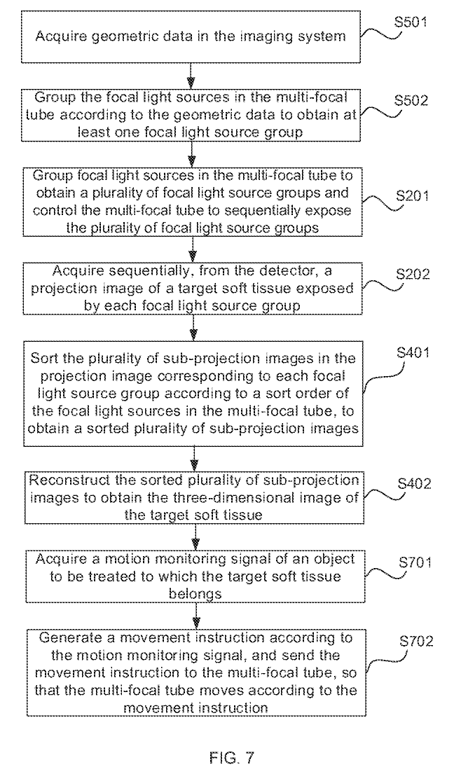

In an implementation, in combination with FIG. 5 and as shown in FIG. 7, the imaging method provided by the embodiments of the present disclosure further includes: S701 to S702.

S701, the imaging control processing device acquires a motion monitoring signal of an object to be treated to which the target soft tissue belongs.

Specifically, since the target soft tissue of the object to be treated may change in position or size, with the movement (such as breathing, heartbeat, voluntary or involuntary bodily movement, etc.) of the object to be treated, the imaging control processing device may monitor the motion monitoring signal of the object to be treated in real time.

In some embodiments, the above-mentioned motion monitoring signal may be a breathing signal of the object to be treated, or a movement signal of the object to be treated (such as a heartbeat signal or other motion signals of the object to be treated, etc.).

S702, the imaging control processing device generates a movement instruction according to the motion monitoring signal, and sends the movement instruction to the multi-focal tube, so that the multi-focal tube moves according to the movement instruction.

As can be seen from the above, since the target soft tissue may change in position or size, with the movement of the object to be treated, it is less effective to move the object to be treated to make the target soft tissue be within the irradiation range of the multi-focal tube. The present disclosure enables, after acquiring the motion monitoring signal of the object to be treated to which the target soft tissue belongs, generating a movement instruction according to the motion monitoring signal, and sending a movement instruction to the multi-focal tube, so that the multi-focal tube moves according to the movement instruction. In this way, both the efficiency and accuracy of moving the multi-focal tube to make the target soft tissue be within the irradiation range of the multi-focal tube, are better than those of moving the object to be treated.

Of course, in the process of acquiring the three-dimensional image of the target soft tissue, the physicist may also need to acquire a three-dimensional image around the target soft tissue. In this case, the physicist may also move the multi-focal tube by the imaging control processing device based on his/her own experiences, and for example, he/she controls the multi-focal tube to be moved to the object to be treated in a same direction or opposite direction by the imaging control processing device, which is not limited in the embodiments of the present disclosure.

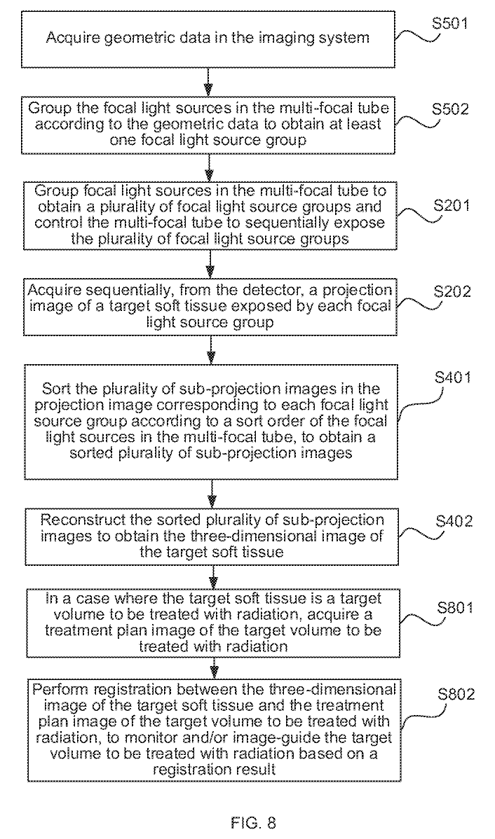

In an implementation, in combination with FIG. 5 and as shown in FIG. 8, the imaging method provided by the embodiments of the present disclosure may also be applied to a radiotherapy system, to set up the patient or monitor the patient in real time in the radiotherapy. The imaging method includes: S801 to S802.

S801, in a case where the target soft tissue is a target volume to be treated with radiation, the imaging control processing device acquires a treatment plan image of the target volume to be treated with radiation.

In an implementation, the imaging control processing device may acquire the treatment plan image of the target volume to be treated with radiation from a treatment plan. The plan image may be a CT (Computed Tomography) image, a magnetic resonance image, or other types of images, which is not limited in the embodiments of the present disclosure.

S802, the imaging control processing device performs registration between the three-dimensional image of the target soft tissue and the treatment plan image of the target volume to be treated with radiation, to set up and/or monitor the target volume to be treated with radiation based on a registration result.

The above introduces the solutions of the embodiments of the present disclosure mainly from the perspective of methods. It can be understood that, in order to implement the above functions, the imaging control processing device contains a corresponding hardware structure and/or software module for performing various functions. Those skilled in the art should easily recognize that, the embodiments of the present disclosure can be implemented in the form of hardware or a combination of hardware and computer software, in combination with the units and algorithm steps of the various examples described in the embodiments disclosed herein. Whether a certain function is performed by hardware or by computer software driving hardware, depends on a specific application and a design constraint condition of the technical solutions. Professional technicians may use different methods to implement the described functions, for each specific application, but this implementation should not be considered beyond the scope of the embodiments of the present disclosure.

In an implementation, the multi-focal tube in the present disclosure may be used compatibly in conjunction with a traditional single-light source tube, to provide users with more options, which improves the convenience and accuracy of treating the target volume to be treated with radiation.

The embodiments of the present disclosure may divide the imaging control processing device into functional units according to the above method examples. For example, various functional units may be divided corresponding to the respective functions, or two or more functions may be integrated into a processing unit. The above integrated unit may be implemented in the form of hardware or in the form of a software functional unit. It should be noted that the division of the units in the embodiments of the present disclosure is schematic, which is only a logical functional division, and there may be other divisions in actual implementations.

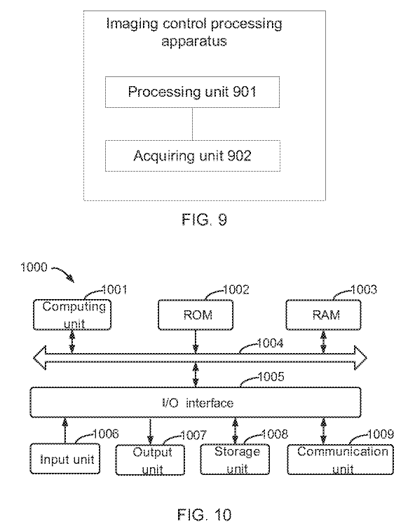

As shown in FIG. 9, the embodiments of the present disclosure provide an imaging control processing apparatus, which may be applied to the imaging control processing device, and is used to control an imaging system to perform imaging. The imaging system includes a multi-focal tube and a detector arranged opposite to the multi-focal tube; the multi-focal tube includes a plurality of focal light sources; the imaging control processing apparatus includes: a processing unit 901 and an acquiring unit 902;

the processing unit 901 is configured to group the focal light sources in the multi-focal tube and control the multi-focal tube to sequentially expose respective focal light source groups, where each focal light source group includes at least one focal light source;

the acquiring unit 902 is configured to acquire sequentially, from the detector, a projection image of a target soft tissue exposed by each focal light source group, where the projection image includes a sub-projection image of the target soft tissue irradiated by each focal light source in the focal light source group corresponding to the projection image, and image contents of sub-projection images in the projection image do not overlap; and

the processing unit 901 is further configured to reconstruct a plurality of sub-projection images in the projection image corresponding to each focal light source group to obtain a three-dimensional image of the target soft tissue.

In some embodiments, the processing unit 901 is specifically configured to:

sort the plurality of sub-projection images in the projection image corresponding to each focal light source group according to a sort order of the focal light sources in the multi-focal tube, to obtain a sorted plurality of sub-projection images; and

reconstruct the sorted plurality of sub-projection images to obtain the three-dimensional image of the target soft tissue.

In some embodiments, each focal light source group satisfies conditions that: the image contents of the sub-projection images of the target soft tissue irradiated by each focal light source in the focal light source group do not overlap, and a number of focal light sources in the focal light source group is greater than a preset number, and a soft tissue irradiation range of each focal light source in the focal light source group covers the target soft tissue, and a range error between the soft tissue irradiation range of each focal light source in the focal light source group and a soft tissue range of the target soft tissue is less than a preset range.

In some embodiments, the processing unit 901 is specifically configured to:

acquire geometric data in the imaging system, where the geometric data includes a linear distance between each focal light source and a center point of the target soft tissue, a vertical distance between the target soft tissue and the detector, a shape and a size of the target soft tissue, and a distance between two focal light sources of the plurality of focal light sources; and

group the focal light sources in the multi-focal tube according to the geometric data to obtain the at least one focal light source group.

In some embodiments, the acquiring unit 902 is further configured to acquire reconstruction time for reconstructing the three-dimensional image of the target soft tissue; and

the processing unit 901 is further configured to add the reconstruction time to the three-dimensional image to obtain a four-dimensional image of the target soft tissue.

In some embodiments, the acquiring unit 902 is further configured to acquire a motion monitoring signal of an object to be treated to which the target soft tissue belongs; and

the processing unit 901 is further configured to generate a movement instruction according to the motion monitoring signal, and send the movement instruction to the multi-focal tube, so that the multi-focal tube moves according to the movement instruction.

In some embodiments, the acquiring unit 902 is further configured to acquire a treatment plan image of the target volume to be treated with radiation, in a case where the target soft tissue is a target volume to be treated with radiation; and

the processing unit 901 is further configured to perform registration between the three-dimensional image of the target soft tissue and the treatment plan image of the target volume to be treated with radiation, to monitor and/or image-guide the target volume to be treated with radiation based on a registration result.

According to the embodiments of the present disclosure, the present disclosure further provides an electronic device, including at least one processor and a memory communicatively connected to the at least one processor; where the memory stores instructions executable by the at least one processor, and the instructions are executed by the at least one processor, to cause the at least one processor to perform the imaging method provided by the present disclosure.

According to the embodiments of the present disclosure, the present disclosure further provides a non-transitory computer readable storage medium storing computer instructions, where the computer instructions are used to cause an electronic device to perform the imaging method provided by the present disclosure.

According to the embodiments of the present disclosure, the present disclosure further provides a computer program product, including a computer program, and the computer program, when executed by a processor, implements the imaging method provided by the present disclosure.

FIG. 10 shows a schematic block diagram of an example electronic device 1000 that may be used to implement the embodiments of the present disclosure. The electronic device is intended to represent various forms of digital computers, such as laptop computers, desktop computers, worktables, personal digital assistants, servers, blade servers, mainframe computers, and other appropriate computers. The electronic device may also represent various forms of mobile apparatuses, such as personal digital assistants, cell phones, smart phones, wearable devices, and other similar computing apparatuses. Herein, the shown components, connections and relationships between these components, and functions of these components are taken as examples only, and are not intended to limit the implementations of the present disclosure as described and/or claimed herein. In some embodiments, the electronic device may be the imaging control processing device shown in FIG. 1 above.

As shown in FIG. 10, the electronic device 1000 includes a computing unit 1001, which may perform various appropriate actions and processes according to a computer program stored in a read-only memory (ROM) 1002 or a computer program loaded to a random access memory (RAM) 1003 from a storage unit 1008. Various programs and data required for the operations of the electronic device 1000 may also be stored in the random access memory (RAM) 1003. The computing unit 1001, the read-only memory 1002, and the RAM 1003 are connected to each other via a bus 1004. An input/output (I/O) interface 1005 is also connected to the bus 1004.

A plurality of components in the electronic device 1000 are connected to the input/output interface 1005. The plurality of components include: an input unit 1006, such as a keyboard, a mouse, etc.; an output unit 1007, such as various types of displays, speakers, etc.; a storage unit 1008, such as a magnetic disk, an optical disk, etc.; and a communication unit 1009, such as a network card, a modem, a wireless communication transceiver, etc. The communication unit 1009 allows the electronic device 1000 to exchange information/data with other devices over a computer network and/or various telecommunication networks such as the Internet.

The computing unit 1001 may be a variety of general-purpose and/or special-purpose processing components with processing and computing capabilities. Some examples of the computing unit 1001 include, but are not limited to, a central processing unit, a graphics processing unit (GPU), various special-purpose artificial intelligence (AI) computing chips, various computing units for executing machine learning model algorithms, a digital signal processor, and any appropriate processor, controller and microcontroller, etc. The computing unit 1001 performs the various methods and processes described above, such as the imaging method. For example, in an embodiment, the imaging method may be implemented as a computer software program, which is tangibly included in a machine-readable medium, such as the storage unit 1008. In an embodiment, a part or all of the computer program may be loaded and/or installed onto the electronic device 1000 via the ROM 1002 and/or the communication unit 1009. When the computer program is loaded onto the RAM 1003 and executed by the computing unit 1001, one or more steps of the imaging method described above may be performed. Alternatively, in other embodiments, the computing unit 1001 may be configured to execute the imaging method in any other appropriate manners (e.g., by means of firmware).

The various implementations of systems and techniques described above herein may be implemented in a digital electronic circuitry system, an integrated circuitry system, a field-programmable gate array, an application-specific integrated circuit, an application-specific standard product (Application Specific Standard Part, ASSP), a system on a chip (SOC) system, a complex programmable logic device (CPLD), computer hardware, firmware, software and/or any combination thereof. The various implementations may include implementations in one or more computer programs. The one or more computer programs may be executed and/or interpreted on a programmable system including at least one programmable processor. The programmable processor may be a special-purpose or general-purpose programmable processor, may receive data and instructions from a storage system, at least one input apparatus and at least one output apparatus, and transmit the data and instructions to the storage system, the at least one input apparatus and the at least one output apparatus.

Program codes for the implementation of the method of the present disclosure may be written in any combination of one or more programming languages. These program codes may be provided for a processor or controller of a general-purpose computer, a special-purpose computer or other programmable data processing apparatuses, to cause functions/operations specified in the flowcharts and/or block diagrams to be implemented when the program codes are executed by the processor or controller. The program codes may all be executed on a machine, or partially be executed on a machine, or partially be executed on a machine and partially be executed on a remote machine as a separate software package, or all be executed on a remote machine or a server.

In the context of the present disclosure, the machine-readable medium may be a tangible medium that contains or stores a program used for an instruction execution system, apparatus or device, or a program used in conjunction with an instruction execution system, apparatus or device. The machine-readable media may be machine-readable signal media or machine-readable storage media. The machine-readable medium may include, but be not limited to, an electronic, magnetic, optical, electromagnetic, infrared or semi-conductive system, apparatus or device, or any appropriate combination thereof. More specific examples of the machine-readable storage medium may include an electrical connection based on one or more wires, a portable computer disk, a hard disk, a random-access memory, a read-only memory, an erasable programmable read-only memory, an optical fiber, a portable compact disc read-only memory, an optical storage device, a magnetic storage device, or any appropriate combination thereof.

In order to provide interactions with a user, the systems and techniques described herein may be implemented on a computer. The computer has: a display apparatus (for example, a cathode ray tube (CRT) or liquid crystal display (LCD) monitor) for displaying information to the user; and a keyboard and a pointing apparatus (for example, a mouse or a trackball), by which the user may provide an input to the computer. Other types of apparatuses may also be used to provide interactions with the user. For example, feedback provided for the user may be sensory feedback in any form (for example, visual feedback, auditory feedback or haptic feedback). Moreover, an input (including sound input, voice input or haptic input) from the user may be received in any form.

The systems and techniques described herein may be implemented in a computing system including a back-end component (for example, as a data server), a computing system including a middleware component (for example, an application server), a computing system including a front-end component (for example, a client computer with a graphical user interface or a web browser, by which the user may interact with implementations of the systems and techniques described herein), or a computing system including any combination of such a back-end component, middleware component or front-end component. The components of the system may be interconnected by any form or medium of digital data communication (for example, a communication network). Examples of the communication network include: a local area network (LAN), a wide area network (WAN) and the Internet.

The computing system may include a client and a server. The client and the server are generally remote from each other and typically interact via a communication network. A relationship between the client and the server is generated by computer programs that are running on the respective computers and have a client-server relationship with each other. The server may be a cloud server, a server of a distributed system, or a server combined with blockchains.

It should be understood that, the various forms of flows shown above may be used, with steps reordered, added or removed. For example, the various steps recorded in the present disclosure may be executed in parallel, in sequence or in a different order, as long as a desired result of the technical solutions of the present disclosure is achieved, which is not limited herein.

The above specific implementations do not constitute a limitation on the protection scope of the present disclosure. It should be understood by those skilled in the art that various modifications, combinations, sub-combinations and substitutions may be made according to design requirements and other factors. Any modifications, equivalent substitutions, improvements and the like made within the spirit and principle of the present disclosure shall fall within the protection scope of the present disclosure.

What is claimed is:

1. An imaging method, wherein the imaging method is used to control an imaging system to perform imaging, the imaging system comprises a multi-focal tube and a detector arranged opposite to the multi-focal tube; the multi-focal tube comprises a plurality of focal light sources; the imaging method comprises:
grouping the focal light sources in the multi-focal tube to obtain a plurality of focal light source groups and controlling the multi-focal tube to sequentially expose the plurality of focal light source groups, wherein each focal light source group of the plurality of focal light source groups comprises at least one focal light source;
acquiring sequentially, from the detector, a projection image of a target soft tissue exposed by each focal light source group, wherein the projection image comprises a sub-projection image of the target soft tissue irradiated by each focal light source in the focal light source group corresponding to the projection image, and image contents of sub-projection images in the projection image do not overlap; and
reconstructing a plurality of sub-projection images in the projection image corresponding to each focal light source group to obtain a three-dimensional image of the target soft tissue.

2. The imaging method according to claim 1, wherein reconstructing the plurality of sub-projection images in the projection image corresponding to each focal light source group to obtain the three-dimensional image of the target soft tissue, comprises:
sorting the plurality of sub-projection images in the projection image corresponding to each focal light source group according to a sort order of the focal light sources in the multi-focal tube, to obtain a sorted plurality of sub-projection images; and
reconstructing the sorted plurality of sub-projection images to obtain the three-dimensional image of the target soft tissue.

3. The imaging method according to claim 1, wherein each focal light source group satisfies conditions that: the image contents of the sub-projection images of the target soft tissue irradiated by each focal light source in the focal light source group do not overlap, and a number of focal light sources in the focal light source group is greater than a preset number, and a soft tissue irradiation range of each focal light source in the focal light source group covers the target soft tissue, and a range error between the soft tissue irradiation range of each focal light source in the focal light source group and a soft tissue range of the target soft tissue is less than a preset range.

4. The imaging method according to claim 3, wherein grouping the focal light sources in the multi-focal tube, comprises:
acquiring geometric data in the imaging system; wherein the geometric data comprises: a linear distance between each focal light source and a center point of the target soft tissue, a vertical distance between the target soft tissue and the detector, a shape and a size of the target soft tissue, and a distance between two focal light sources of the plurality of focal light sources; and
grouping the focal light sources in the multi-focal tube according to the geometric data to obtain at least one focal light source group.

5. The imaging method according to claim 1, wherein the imaging method further comprises:
acquiring reconstruction time for reconstructing the three-dimensional image of the target soft tissue; and
adding the reconstruction time to the three-dimensional image to obtain a four-dimensional image of the target soft tissue.

6. The imaging method according to claim 1, wherein the imaging method further comprises:
acquiring a motion monitoring signal of an object to be treated to which the target soft tissue belongs; and
generating a movement instruction according to the motion monitoring signal, and sending the movement instruction to the multi-focal tube, so that the multi-focal tube moves according to the movement instruction.

7. The imaging method according to claim 1, wherein the imaging method further comprises:
in a case where the target soft tissue is a target volume to be treated with radiation, acquiring a treatment plan image of the target volume to be treated with radiation; and
performing registration between the three-dimensional image of the target soft tissue and the treatment plan image of the target volume to be treated with radiation, to set up and/or monitor the target volume to be treated with radiation based on a registration result.

8. An imaging system, comprising:
a gantry;
a multi-focal tube, the multi-focal tube comprising a plurality of focal light sources;
a detector arranged opposite to the multi-focal tube; and
an imaging control processing device, configured to perform operations of:
grouping the focal light sources in the multi-focal tube to obtain a plurality of focal light source groups and controlling the multi-focal tube to sequentially expose the plurality of focal light source groups, wherein each focal light source group of the plurality of focal light source groups comprises at least one focal light source;
acquiring sequentially, from the detector, a projection image of a target soft tissue exposed by each focal light source group, wherein the projection image comprises a sub-projection image of the target soft tissue irradiated by each focal light source in the focal light source group corresponding to the projection image, and image contents of sub-projection images in the projection image do not overlap; and
reconstructing a plurality of sub-projection images in the projection image corresponding to each focal light source group to obtain a three-dimensional image of the target soft tissue.

9. The imaging system according to claim 8, wherein the imaging system further comprises a plurality of beam limiters corresponding one-to-one to the plurality of focal light sources; an aperture direction of a beam limiter of the plurality of beam limiters is a preset direction so that a central ray direction of a focal light source corresponding to the beam limiter is a direction of the target soft tissue; an aperture size of the beam limiter is a preset size so that a range error between a soft tissue irradiation range of the focal light source corresponding to the beam limiter and a soft tissue range of the target soft tissue is less than a preset range.

10. The imaging system according to claim 8, wherein reconstructing the plurality of sub-projection images in the projection image corresponding to each focal light source group to obtain the three-dimensional image of the target soft tissue, comprises:
sorting the plurality of sub-projection images in the projection image corresponding to each focal light source group according to a sort order of the focal light sources in the multi-focal tube, to obtain a sorted plurality of sub-projection images; and
reconstructing the sorted plurality of sub-projection images to obtain the three-dimensional image of the target soft tissue.

11. The imaging system according to claim 8, wherein each focal light source group satisfies conditions that: the image contents of the sub-projection images of the target soft tissue irradiated by each focal light source in the focal light source group do not overlap, and a number of focal light sources in the focal light source group is greater than a preset number, and a soft tissue irradiation range of each focal light source in the focal light source group covers the target soft tissue, and a range error between the soft tissue irradiation range of each focal light source in the focal light source group and a soft tissue range of the target soft tissue is less than a preset range.

12. The imaging system according to claim 11, wherein grouping the focal light sources in the multi-focal tube, comprises:
acquiring geometric data in the imaging system; wherein the geometric data comprises: a linear distance between each focal light source and a center point of the target soft tissue, a vertical distance between the target soft tissue and the detector, a shape and a size of the target soft tissue, and a distance between two focal light sources of the plurality of focal light sources; and
grouping the focal light sources in the multi-focal tube according to the geometric data to obtain at least one focal light source group.

13. The imaging system according to claim 8, wherein the imaging control processing device is further configured to:
acquire reconstruction time for reconstructing the three-dimensional image of the target soft tissue; and
add the reconstruction time to the three-dimensional image to obtain a four-dimensional image of the target soft tissue.

14. The imaging system according to claim 8, wherein the imaging control processing device is further configured to:
acquire a motion monitoring signal of an object to be treated to which the target soft tissue belongs; and
generate a movement instruction according to the motion monitoring signal, and sending the movement instruction to the multi-focal tube, so that the multi-focal tube moves according to the movement instruction.

15. The imaging system according to claim 8, wherein the imaging control processing device is further configured to:
in a case where the target soft tissue is a target volume to be treated with radiation, acquire a treatment plan image of the target volume to be treated with radiation; and
perform registration between the three-dimensional image of the target soft tissue and the treatment plan image of the target volume to be treated with radiation, to set up and/or monitor the target volume to be treated with radiation based on a registration result.

16. An electronic device, wherein the electronic device comprises:
a processor; and
a memory configured to store instructions executable by the processor;
wherein the processor is configured to execute the instructions to implement operations of:
grouping the focal light sources in the multi-focal tube to obtain a plurality of focal light source groups and controlling the multi-focal tube to sequentially expose the plurality of focal light source groups, wherein each focal light source group of the plurality of focal light source groups comprises at least one focal light source;
acquiring sequentially, from the detector, a projection image of a target soft tissue exposed by each focal light source group, wherein the projection image comprises a sub-projection image of the target soft tissue irradiated by each focal light source in the focal light source group corresponding to the projection image, and image contents of sub-projection images in the projection image do not overlap; and
reconstructing a plurality of sub-projection images in the projection image corresponding to each focal light source group to obtain a three-dimensional image of the target soft tissue.

17. The electronic device according to claim 16, wherein the processor is further configured to:
sort the plurality of sub-projection images in the projection image corresponding to each focal light source group according to a sort order of the focal light sources in the multi-focal tube, to obtain a sorted plurality of sub-projection images; and
reconstruct the sorted plurality of sub-projection images to obtain the three-dimensional image of the target soft tissue.

18. The electronic device according to claim 16, wherein the processor is further configured to:
acquire reconstruction time for reconstructing the three-dimensional image of the target soft tissue; and
add the reconstruction time to the three-dimensional image to obtain a four-dimensional image of the target soft tissue.

19. The electronic device according to claim 16, wherein the processor is further configured to:
acquire a motion monitoring signal of an object to be treated to which the target soft tissue belongs; and
generate a movement instruction according to the motion monitoring signal, and sending the movement instruction to the multi-focal tube, so that the multi-focal tube moves according to the movement instruction.

20. The electronic device according to claim 16, wherein the processor is further configured to:
in a case where the target soft tissue is a target volume to be treated with radiation, acquire a treatment plan image of the target volume to be treated with radiation; and
perform registration between the three-dimensional image of the target soft tissue and the treatment plan image of the target volume to be treated with radiation, to set up and/or monitor the target volume to be treated with radiation based on a registration result.
